SEQUENCE LISTING (17 sequences)

SEQ ID NO: 1 - DNA, 318 bp, Mus sp.
caaattgttctctcccagtctccagcaatcctgtctgcatctccaggggagaaggtcacaatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttattctttcacaatcagcagagtggaggctgaagatgctgccacttattactgccagcagtggacttttaacccacccacgttcggaggggggaccaggctggaaataaaa

SEQ ID NO: 2 - DNA, 354 bp, Mus sp.
caggcttatctacagcagtctggggctgagctggtgaggcctggggcctcagtgaagatgtcctgcaaggcttctggctacacatttaccagttacaatatgcactgggtaaagcagacacctagacagggcctggaatggattggaggtatttatccaggaaatggtgatacttcctacaatcagaagttcaagggcaaggccacactgactgtaggcaaatcctccagcacagcctacatgcagctcagcagcctgacatctgaagactctgcggtctatttctgtgcaagatatgactacaactatgctatggactactggggtcaaggaacctcagtcaccgtctcctca

SEQ ID NO: 3 - DNA, 990 bp, Homo sapiens
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 4 - DNA, 321 bp, Homo sapiens
cggactgtggctgcaccaagtgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 5 - DNA, 639 bp, Artificial
  chimeric mice (Mus sp.) - Humain (Homo sapiens)
caaattgttctctcccagtctccagcaatcctgtctgcatctccaggggagaaggtcacaatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttattctttcacaatcagcagagtggaggctgaagatgctgccacttattactgccagcagtggacttttaacccacccacgttcggaggggggaccaggctggaaataaaacggactgtggctgcaccaagtgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 6 - DNA, 1344 bp, Artificial
  Chimeric mice (Mus sp.) - Humain (Homo sapiens)
caggcttatctacagcagtctggggctgagctggtgaggcctggggcctcagtgaagatgtcctgcaaggcttctggctacacatttaccagttacaatatgcactgggtaaagcagacacctagacagggcctggaatggattggaggtatttatccaggaaatggtgatacttcctacaatcagaagttcaagggcaaggccacactgactgtaggcaaatcctccagcacagcctacatgcagctcagcagcctgacatctgaagactctgcggtctatttctgtgcaagatatgactacaactatgctatggactactggggtcaaggaacctcagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 7 - protein, 213 aa, Artificial
  chimeric mice (Mus sp.) - Humain (Homo sapiens)
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 448 aa, Artificial
  chimeric mice (Mus sp.) - Humain (Homo sapiens)
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 24 bp, Artificial
  reverse transcription primers Kappa murine specific reverse primer
actgccatcaatcttccacttgac

SEQ ID NO: 10 - DNA, 23 bp, Artificial
  reverse transcription primers G2a murine specific reverse primer
ctgagggtgtagaggtcagactg

SEQ ID NO: 11 - DNA, 28 bp, Artificial
  5′RACE PCR primers Kappa murine specific reverse primer
ttgttcaagaagcacacgactgaggcac

SEQ ID NO: 12 - DNA, 28 bp, Artificial
  5′RACE PCR primers G2a murine specific reverse primer
gagttccaggtcaaggtcactggctcag

SEQ ID NO: 13 - DNA, 47 bp, Artificial
  Kappa light chain vector of the antibody EMAB603  forward VK primer
ctcagtactagtgccgccaccatggattttcaagtgcagattttcag

SEQ ID NO: 14 - DNA, 46 bp, Artificial
  Kappa light chain vector of the antibody EMAB603  reverse VK primer
tgaagacacttggtgcagccacagtccgttttatttccagcctggt

SEQ ID NO: 15 - DNA, 45 bp, Artificial
  Heavy chain vector  forward VH primer
ctcagtactagtgccgccaccatgggattcagcaggatctttctc

SEQ ID NO: 16 - DNA, 48 bp, Artificial
  Heavy chain vector reverse VH primer
gaccgatgggcccttggtggaggctgaggagacggtgactgaggttcc

SEQ ID NO: 17 - DNA, 11141 bp, Artificial
  plasmide HK463-25 crude sequence
gatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagcttggtaccgagctcggatccactagagcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttgctagcgccgccaccatgggattcagcaggatctttctcttcctcctgtcagtaactacaggtgtccactcccaggcttatctacagcagtctggggctgagctggtgaggcctggggcctcagtgaagatgtcctgcaaggcttctggctacacatttaccagttacaatatgcactgggtaaagcagacacctagacagggcctggaatggattggaggtatttatccaggaaatggtgatacttcctacaatcagaagttcaagggcaaggccacactgactgtaggcaaatcctccagcacagcctacatgcagctcagcagcctgacatctgaagactctgcggtctatttctgtgcaagatatgactacaactatgctatggactactggggtcaaggaacctcagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatagggcgcgccgctagagaggatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttcatatgctcgagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagcttggtaccgagctcggatccactagagcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttactagtgccgccaccatggattttcaagtgcagattttcagcttcctgctaatcagtgcttcagtcataatgtccagaggacaaattgttctctcccagtctccagcaatcctgtctgcatctccaggggagaaggtcacaatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttattctttcacaatcagcagagtggaggctgaagatgctgccacttattactgccagcagtggacttttaacccacccacgttcggaggggggaccaggctggaaataaaacggactgtggctgcaccaagtgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtgaactctagagaggatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttcatatgctcgagatttaaatactggggctcgactgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggggggggggacagctcagggctgcgatttcgcgccaaacttgacggcaatcctagcgtgaaggctggtaggattttatccccgctgccatcatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatcgatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagagatctcgagggtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatcgatcctctagctagagtcgatcgacctgcagggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagagatcttagatatccggacgtgtataccagtttaaacatgttaattaagtcgacgcggccgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgaca

(19) United States
(12) Patent Application Publication
(10) Pub. No.: US 2012/0100133 A1
(43) Pub. Date: Apr. 26, 2012

(54) USE OF ANTI-CD20 ANTIBODY FOR TREATING PRIMARY INTRAOCULAR LYMPHOMA

(75) Inventors: Sylvain Fisson, La Queue En Brie (FR); Catherine Fridman, Paris (FR); Remi Urbain, L'Hay Les Roses (FR)

(73) Assignee: LFB BIOTECHNOLOGIES, Les Ulis (FR)

(21) Appl. No.: 13/143,056

(22) PCT Filed: Nov. 26, 2009
(86) PCT No.: PCT/FR2009/001349

§ 371 Date: Nov. 2, 2011

(30) Foreign Application Priority Data
Dec. 30, 2008 (FR) 0807496

(51) Int. Cl. A61K 39/395; A61P 35/00; A61P 27/02; C07K 16/46

(52) U.S. Cl. 424/133.1; 530/387.3

(57) ABSTRACT

An embodiment relates to a monoclonal antibody directed against the CD20 antgen, in which the variable region of each of the light chains is coded by murine nucleic acid sequence SEQ ID NO:1, the variable region of each of the heavy chains is coded by murine nucleic acid sequence SEQ ID NO: 2, and the constant regions of the light chains and of the heavy chains originate from a non-murine species, said antibody being used for treating primary intraocular lymphoma.

### PRIORITY CLAIM

The present application is a national phase application filed pursuant to 35 USC §371 of International Patent Application Serial No. PCT/FR2009/001349, filed Nov. 26, 2009; which further claims the benefit of French Patent Application Serial No. 08/07496 filed Dec. 30, 2008; all of the foregoing applications are incorporated herein by reference in their entireties.

### TECHNICAL FIELD

An embodiment relates to a monoclonal antibody directed against the CD20 antigen, in which the variable region of each of the light chains is coded by murine nucleic acid sequence SEQ ID NO: 1 in which the variable region of each of the heavy chains is coded by murine nucleic acid sequence SEQ ID NO: 2, and in which the constant regions of the light chains and the heavy chains originate from a non-murine species, as a drug for treating primary intraocular lymphoma (also called “PIOL”).

In the description below, the references between brackets ([ ]) refer to the list of references given after the examples.

### Sequence Listing

The material submitted in the accompanying Sequence Listing is incorporated herein by reference in its entirety. The Sequence Listing is submitted as an ASCII compliant text file with a filename of 2978-001-03_SequenceListing_{—}20120103.txt, a creation date of Jan. 3, 2012, and a text file size of 28 kilobytes. The Sequence Listing does not include any new matter that goes beyond the disclosure of the present application.

### BACKGROUND

The CD20 antigen is a hydrophobic transmembrane protein with a molecular weight of 35-37 kDa present on the surface of mature B lymphocytes (Valentine et al. (1987) Proc Natl Acad Sci USA. 84(22): 8085-9 [1] Valentine et al. (1989) J. Biol. Chem. 264(19): 11282-11287 [2], which are incorporated by reference). It is expressed during the development of B lymphocytes from the early pre-B stage until differentiation into plasmocyte, a stage at which this expression disappears. The CD20 antigen is present on both normal B lymphocytes and malignant B cells. More particularly, the CD20 antigen is expressed on most phenotype-B lymphomas (80% of lymphomas): for instance, it is expressed on over 90% of non-Hodgkin's B-lymphocytes lymphomas (NHL).

The function of CD20 has not yet been fully clarified, but it may act as a calcium channel and be involved in the regulation of the first stages of B lymphocytes differentiation (Golay et al. (1985) J. Immunol.; 135(6): 3795-801 [3]) and proliferation (Tedder et al. (1986) Sur J. Immunol. 1986 August; 16(8): 881-7 [4], which are incorporated by reference).

Therefore, although uncertainty remains as regards its role in the activation and the proliferation of the B lymphocytes, the CD20 antigen is, because of its location, an important target for the treatment of pathologies involving tumor B lymphocytes, such as NHL or B-CLL (B-cell chronic lymphocytic leukemia) for example, using antibodies which specifically recognize CD20. In addition, this antigen is an ideal target because it is a membrane protein for which no expression modulation or polymorphism is known.

Only one non-radiolabelled anti-CD20 monoclonal antibody, rituximab (Genentech), is currently available on the market for treating B-cell lymphomas. It shows very encouraging clinical results in patients with NHL when associated with chemotherapy. Yet, its effectiveness remains variable and often modest when it is used as the only agent (Teeling et al. (2004) Blood 104(6).-1793-800[5], which is incorporated by reference).

The primary intraocular lymphoma (PIOL) is a non-Hodgkin lymphoma of the central nervous system. This form generally affects the vitreous or the retina.

PIOL ocular signs may precede central or systemic nervous impairment, sometimes by years, and the ophthalmologist can sometimes be the first to make a diagnosis.

The age at which the first signs appear varies, but it is rare to see this affection before the forties. Generally, patients are over 60. Initially, the impairment is unilateral, but it becomes bilateral in 50 to 80% of cases.

The first ocular outbreaks are generally a reduced visual acuity or a blurred vision.

The diagnosis may be conducted by a cytological control of the posterior chamber puncture, revealing the presence of cells characteristic of a highly-malignant non-Hodgkin lymphoma.

The most commonly used treatment method is chemotherapy by systemic methotrexate or by intravitreal injection. However, treatment by methotrexate requires several injections and leads to ocular complications, such as corneal epitheliopathy (Ohguro et al.(2008).Arch. Ophtalmol/Vol. 126 (No. 7); 1002-1003[6], which is incorporated by reference). Besides, another case reported by SMET MD shows that even when the intravitreously administered methotrexate is generally well tolerated and leads to a quick healing, it does prevent relapse in case of monotherapy (SMET et al. Bull. Soc. Beige Ophtalmol., 279,91-95,2001[20], which is incorporated by reference). In addition, the methotrexate is known for having a number of other adverse reactions and particularly an immunosuppression and hepatic impairment.

PIOL is a lymphoma comprising large B cell neoplasms expressing CD20. Intravenous injections of rituximab contributed in prolonging survival of patients with systemic large B cells lymphoma. However, rituximab did not affect the prognosis of the central nervous system lymphoma, probably because the monoclonal antibody does not cross the blood brain barrier. Likewise, it is unlikely that rituximab intravenous injection treatment of eye injuries associated with the central nervous system lymphoma be beneficial because of the blood ocular barrier ([6]).

However, rituximab intravenous injection tests showed absence of significant eye toxic effects in patients with central nervous system primary lymphoma (Kitzamnn et al. (2007) Eye 21, 1524-1527 [7], which is incorporated by reference). Yet, because of complementary systemic or ocular treatments received by patients, it is not possible to draw conclusions with respect to the effectiveness of a therapy by intravenous administration of rituximab.

In addition, tests of intravenous rituximab injections, in two PIOL patients showed a disappearance of the lymphoma malignant cells in the eye ([6]). Yet, half the cases presented an inflammatory reaction of the anterior chamber of the eye. The clinical study also indicates the absence of complications. However, the observations were limited to two months following the treatment by rituximab and they were not based on biopsy results, but solely on clinical observations.

### SUMMARY

There is, therefore, a real need for a therapeutic tool which overcomes these drawbacks and obstacles regarding PIOL treatment.

An embodiment relates to the use of a monoclonal antibody directed against the CD20 antigen, in which the variable region of each of the light chains is coded by murine nucleic acid sequence SEQ ID NO: 1, in which the variable region of each of the heavy chains is coded by murine nucleic acid sequence SEQ ID NO:2, and in which the constant regions of the light chains and the heavy chains originate from a non-murine species, for treating primary intraocular lymphoma (also called “PIOL”).

Antibodies are made of heavy and light chains, linked together by disulphide bonds. Each chain is made up, in the N-terminal position, of a variable region (or domain) (coded by rearranged V-J genes for the light chains and V-D-J genes for the heavy chains) specific to the antigen against which the antibody is directed, and, in the C-terminal position, of a constant region made up of a single CL domain for the light chains or several domains for the heavy chains.

For the purposes of the disclosure, the expressions “monoclonal antibody” or “monoclonal antibody composition” refer to a preparation of antibody molecules having an identical and unique specificity. The antibody according to an embodiment, in which the variable regions of the light and heavy chains belong to a species different from the constant regions of the light and heavy chains, is referred to as a “chimeric” antibody.

Murine nucleic acid sequences SEQ ID NO: 1 and SEQ ID NO:2 code the variable domain of each of the light chains and the variable domain of each of the heavy chains respectively, of the antibody produced by murine hybridoma CAT-13.6E12, available at the Deutsche Sammlung von Mikroorganismen and Zellkulturen GmbH (DSMZ) under number ACC 474. This hybridoma produces a murine IgG2a, k-type monoclonal antibody directed against the CD20.

SEQ ID NO.1 sequence nevertheless includes a nucleic acid which differs from the sequence coding the variable region of the light chain of the antibody produced by CAT-13.6E12 murine hybridoma.

These murine sequences have been chosen to derive the sequences of the variable regions of the antibodies according to an embodiment because of the specificity of the CAT-13.6E12 murine antibody for the CD20 antigen. The variable regions of the antibodies according to an embodiment share at least approximately 70% identity with sequences SEQ ID NO: 1 and SEQ ID NO:2, this sequence identity making the antibodies according to an embodiment to have identical specificity with the CAT-13.6E12 murine antibody. This sequence identity may also provide an identity affinity for the target between the antibody according to an embodiment and the CAT-13.6E12 murine antibody.

In addition, the antibodies used in an embodiment possess constant regions of their light and heavy chains belonging to a non-murine species. In this regard, all families and species of non-murine mammals are likely to be used, and in particular humans, monkeys, murine (apart from mice), porcine, bovine, equine, feline, canine, as well as birds.

The antibodies used as a treatment in an embodiment may be constructed using standard recombinant DNA techniques, which are well-known, and more particularly using the “chimeric” antibodies construction techniques described, for example, in Morrison et al. Proc. Natl. Acad. Sci. USA., 81,pp. 6851-55 (1984), which is incorporated by reference, where the recombinant DNA technology is used to replace the constant region of a heavy chain and/or the constant region of a light chain of an antibody from a nonhuman mammal with the corresponding regions in a human immunoglobulin. A particular embodiment will be illustrated below.

Advantageously, the variable region of each of the light chains of the antibody used in an embodiment is coded by a sequence sharing at least approximately 80% identity with murine nucleic acid sequence SEQ ID NO:1, and the variable region of each of the heavy chains of the antibody according to an embodiment is coded by a sequence sharing at least approximately 80% identity with murine nucleic acid sequence SEQ ID NO:2. Advantageously, the variable region of each of the light chains of the antibody according to an embodiment is coded by a sequence sharing at least approximately 90% identity with murine nucleic acid sequence SEQ ID NO:1 and the variable region of each of the heavy chains of the antibody according to an embodiment is coded by a sequence sharing at least approximately 90% identity with murine nucleic acid sequence SEQ ID NO:2.

Advantageously, the variable region of each of the light chains of the antibody used in an embodiment is coded by a sequence sharing at least approximately 95% identity with murine nucleic acid sequence SEQ ID NO: 1, and the variable region of each of the heavy chains of the antibody according to an embodiment is coded by a sequence sharing at least approximately 95% identity with murine nucleic acid sequence SEQ ID NO:2.

Advantageously, the variable region of each of the light chains of the antibody used in an embodiment is coded by a sequence sharing at least approximately 99% identity with murine nucleic acid sequence SEQ ID NO: 1, and the variable region of each of the heavy chains of the antibody according to an embodiment is coded by a sequence sharing at least approximately 99% identity with murine nucleic acid sequence SEQ ID NO: 2.

Advantageously, any antibody in which the variable regions of the heavy and light chains include one or more substitution(s), insertion(s) or deletion(s) of one or more nucleic acids, with these sequences modifications corresponding to the sequences identity percentage defined above, without affecting the antibody's specificity or affinity for the target, can be used in the context of an embodiment.

The antibodies used in an embodiment also concern any antibody which has the CDR (Complementary Determining Region) regions of the CAT-13.6E12 antibody, combined with the FR (framework, highly conserved regions of the variable regions, also known as “backbone”). Such antibodies have affinities and specificities which are very closely comparable with, and which may be identical to, the CAT-13.6E12 murine antibody.

The variable region of each of the light chains of the antibody used in an embodiment may be coded by murine nucleic acid sequence SEQ ID NO:1, and the variable region of each of the heavy chains of the antibody according to an embodiment is coded by murine nucleic acid sequence SEQ ID NO:2.

In an embodiment, an antibody used, particularly as a drug, is a monoclonal antibody directed against the CD20 antigen in which the variable region of each of the light chains is coded by murine nucleic acid sequence SEQ ID NO:1, the variable region of each of the heavy chains is coded by murine nucleic acid sequence SEQ ID NO:2, and the constant regions of the light chains and heavy chains are constant regions from a non-murine species.

Thus, what is meant by “directed against the CD20 antigen” is the ability of the monoclonal antibody to connect all or part of the CD20 antigen, and particularly, the epitope recognized by the antibody EMAB603.

The constant regions of each of the light chains and each of the heavy chains of the antibody used in an embodiment are human constant regions. This embodiment makes it possible to reduce the immunogenicity of the antibody in humans and at the same time to improve its effectiveness upon therapeutic administration to humans. In an embodiment, the constant region of each of the light chains of the antibody of an embodiment is of K-type. Any allotype is suitable for the implementation of an embodiment, e.g. Km(1), Km(1,2), Km(1,2,3) and Km(3) but in an embodiment a preferred allotype is Km(3).

In another embodiment, the constant region of each of the light chains of the antibody according to an embodiment is of type.

In a particular embodiment, and particularly when the constant regions of each of the light chains and each of the heavy chains of the antibody used are human regions, the constant region of each of the heavy chains of the antibody is of type. According to this alternative, the constant region of each of the heavy chains of the antibody may be 1 type, 2 type, 3 type, with these three constant region types exhibiting the particular feature of binding the human complement, or even of 4 type. Antibodies with a constant region of each heavy chain-type belong to the IgG class. Immunoglobulin type G (IgG) are heterodimers composed of two heavy chains and two light chains, linked together by disulfide bonds. Each chain is composed of, in the N-terminal position, a variable region or domain (coded by rearranged V-J genes for the light chain and V-D-J genes for the heavy chain) specific to the antigen against which the antibody is directed, and, in the C-terminal position, of a constant region, composed of a single CL domain for the light chain or of 3 domains (CH1, CH2 and CH3) for the heavy chain. The combination of the variable domains and the CH1 and CL domains of the heavy and light chains make up the Fab fragments, which are linked to the Fc region through a highly flexible hinge region making it possible for each Fab to bind its antigen target whilst the Fc region, the mediator for the effector properties of the antibody, remains accessible to the effector molecules such as Fc R and C1q receptors. The Fc region, composed of both CH2 and CH3 globular domains, is glycosylated at the CH2 domain with the presence, on each of the two chains, of a lactosamine-type biantennary N-glycan linked to the Asn 297.

The constant region of each of the heavy chains of the antibody may be of the 1 type, because such an antibody exhibits an ability to generate ADCC activity in the greatest number of (human) individuals. In this respect, any allotype is suitable for the implementation of an embodiment, e.g. G1m(3), G1m(1,2,17) G1m(1,17) or G1m(1,3); but in an embodiment, preferably the allotype is G1m (1, 17).

In an embodiment, the constant region of each of the heavy chains of the antibody is 1 type, and is coded by human nucleic acid sequence SEQ ID NO:3, with the constant region of each of its light chains coded by human nucleic acid sequence SEQ ID NO:4. Thus, such an antibody has a murine variable region and human constant region, with 1-type heavy chains. This antibody therefore belongs to the IgGI subclass. According to an embodiment of the antibody used, the antibody has two light chains, in which the variable domain is coded by nucleic acid sequence SEQ ID NO: 1 and human constant region is coded by nucleic acid sequence SEQ ID NO: 4, and two heavy chains, the variable domain of which is coded by murine nucleic acid sequence SEQ ID NO:2 and the constant region is coded by human nucleic acid sequence SEQ ID NO:3.

Each of the light chains of the antibody according to an embodiment may be coded by murine-human chimeric nucleic acid sequence SEQ ID NO:5, and each of the heavy chains may be coded by murine-human chimeric nucleic acid sequence SEQ ID NO:6. Murine-human chimeric nucleic acid sequence SEQ ID NO:5 coding each of the light chains of the antibody is obtained by fusing murine nucleic acid sequence SEQ ID NO:1 which codes the variable domain of each of the light chains of the antibody and human nucleic SEQ ID NO:4 which codes the constant region of each of the light chains of the antibody.

Murine-human chimeric nucleic acid sequence SEQ ID NO:6 coding each of the heavy chains of the antibody is obtained by fusing murine nucleic acid sequence SEQ ID NO:2 coding the variable domain of each of the heavy chains of the antibody and of human nucleic acid sequence SEQ ID NO:3 coding the constant region of each of the heavy chains of the antibody.

In an embodiment, when each of the light chains of the antibody is coded by murine-human chimeric nucleic acid sequence SEQ ID NO: 5, and each one of the heavy chains is coded by murine-human chimeric nucleic acid sequence SEQ ID NO:6, the peptide sequence of each of the light chains, deduced from nucleic acid sequence SEQ ID NO:5 is SEQ ID NO:7 and the peptide sequence of each of the heavy chains, deduced from nucleic acid sequence SEQ ID NO:6 is sequence SEQ ID NO:8.

The amino acid located at position 106 is a lysine (K) in SEQ ID NO: 7 sequence.

An embodiment also relates to antibodies in which each of the light chains coded by a murine-human chimeric nucleic acid sequence shares at least approximately 70% homology or identity with murine-human chimeric nucleic acid sequence SEQ ID NO:7 and each one of the heavy chains coded by a murine-human chimeric nucleic acid sequence which shares at least approximately 70% homology or identity with murine-human chimeric nucleic acid sequence SEQ ID NO:8, these modifications altering neither the specificity of the antibody nor its effector activities, such as ADCC (Antibody-Dependent Cell-Mediated Cytotoxicity) activity.

In a particularly advantageous manner, the antibody used in an embodiment is produced by a rat hybridoma cell line. The line producing the antibody according an embodiment may be an important feature since it provides the antibody with some of its particular properties. In fact, the method of expression of the antibody induces the post-translational modifications, particularly the glycosylation modifications, which may vary from one cell line to another, and thereby provide antibodies which otherwise have identical primary structures with different functional properties.

In an embodiment, the antibody is produced in the rat hybridoma YB2/0 cell line (YB2/3HL.P2.G11.16Ag.2O, registered at the American type Culture Collection under number ATCC CRL-1662). This line was chosen because of its ability to produce antibodies with improved ADCC activity compared to antibodies with the same primary structure produced, for example, in CHO cells.

In another embodiment, another antibody is EMAB603 antibody produced by clone R603 registered on Nov. 29, 2005 under registration number CNCM 1-3529 at the Collection Nationale de Cultures de Microoganismes (CNCM, Institut Pasteur, 25 rue du Docteur Roux, 75724 Paris Cedex 15). Each of the light chains of the EMAB603 antibody is coded by murine-human chimeric nucleic acid sequence SEQ ID NO: 5, and each of the heavy chains is coded by murine-human chimeric nucleic acid sequence SEQ ID NO:6. This chimeric antibody competes with CAT-13.6E12 murine antibody in binding CD20 and has a cytotoxic activity much greater than that of rituximab, which may be attributable in part to the specific glycosylation of the N-glycan of the heavy chain of these antibodies. In fact, a particular feature of the R603 clone is that it produces an EMAB603 antibody composition with a fucose/galactose ratio less than 0.6, for which it has been shown in patent application FR 03 12229, which is incorporated by reference, to be optimal to provide the antibody with strong ADCC activity. This antibody may be particularly useful as a therapeutic tool for the treatment of PIOL.

Advantageously, the antibodies of an embodiment may allow the formation of memory T lymphocytes. These memory T lymphocytes may possess the capacity to proliferate and to reactivate quickly during a second exposure to tumor cells.

Advantageously, the antibodies of an embodiment may allow infiltration of T cells in the tumor, which may lead to a slowing of tumor growth, and an improved clearance of tumor cells.

Advantageously, such a monoclonal antibody may have a significant antitumor effect. For example, such an antibody may allow inhibition of the replication of tumor cells by at least approximately 20%, or at least approximately 30%, or at least approximately 50%, or, for example, at least approximately 60% or 70% in the patient with intraocular lymphoma to whom it is administered.

Such antibodies, as well as a method for producing them, are described in WO 2006/064121, which is incorporated by reference.

One example of an antibody expression vector according to an embodiment is the SEQ ID NO: 17 sequence vector. This vector allows the expression of an antibody according to an embodiment, in which the light chain is coded by nucleic acid sequence SEQ ID NO: 5, in which the deduced peptide sequence is SEQ ID NO: 7, and in which the heavy chain is coded by nucleic acid sequence SEQ ID NO: 6, and in which the deduced peptide sequence is SEQ ID NO: 8. This vector is a nucleic acid molecule in which nucleic acid sequence SEQ ID NO: 5 coding each of the light chains of the antibody and nucleic acid sequence SEQ ID NO: 6 coding each of the heavy chains of the antibody have been inserted to be introduced and maintained in a host cell. It allows the expression of these foreign nucleic acid fragments into the host cell since it has sequences (promoter, polyadenylation sequence, gene selection) which are essential to this expression. Such vectors are well known and may, in a non-exhaustive way, be an adenovirus, a retrovirus, a plasmid or a bacteriophage. In addition, any mammalian cell can be used as a host cell, that is as a cell which expresses the antibody according to an embodiment, example YB2/0, CHO, CHO-dhfr- (for example CHO DX BII, CHO DG44), Lec13 CHO, SP2/0, NSO, 293, BHK or COS.

In an embodiment, the antibody can be produced by co-expression in a two-expression vectors cell, one allowing the expression of the light chain and the other the expression of the heavy chain of the antibody. As noted above, these vectors have sequences (promoter, polyadenylation sequence, gene selection) which are essential to this expression. As indicated earlier, the vectors may be, for example, a plasmid, an adenovirus, a retrovirus or a bacteriophage, and the host cell may be any mammalian cell, example YB2/0, CHO, CHO-dhfr (CHO DX BII, CHO DG44) Lecl3 CHO, SP2/0, NSO, 293, BHK or COS.

Advantageously, the stable cell line which expresses an antibody used in an embodiment is selected from the group consisting of: SP2/0, YB2/0, IR983F, a human myeloma such as Namalwa or any other cell of human origin such as PERC6, CHO lines, particularly CHO-K-1, CHO-Lec10, CHO-Lec1, CHO-Lec13, CHO Pro-5, CHO dhfr-(DX BII CHO, CHO DG44), or other lines selected from Wil-2, Jurkat, Vero, Molt-4, COS-7, 293-HEK, BHK, K6H6, NSO, SP2/0-Ag 14 and P3X63Ag8.653.

Preferably, the line used is the rat hybridoma YB2/0 cell line (YB2/3HL.P2.G11.Ag.2O cell, registered at the American Type Culture Collection under number ATCC CRL-1662). This line was chosen because of its ability to produce antibodies with improved ADCC activity compared to the same primary structure of antibodies produced in CHO, for example.

The culture medias suitable for these cells are well known and include, in a non exhaustive manner, the RPMI 1640 culture media (The Journal of the American Medical Association, 199, 519 (1967) [14], which is incorporated by reference), Eagle's MEM (Science, 122, 501 (1952) [15], which is incorporated by reference), Dulbecco's modified MEM (Virology, 8, 396 (1959) [16], which is incorporated by reference), F12 Medium (Proc. Natl. Acad. Sci. USA, 53, 288 (1965) [17], which is incorporated by reference), IMDM (J. Experimental Medicine, 147, 923(1978) [18], which is incorporated by reference), or those described in patent EP1229125, which is incorporated by reference.

An embodiment relates to a method of treatment using the previously described antibodies of primary intraocular lymphoma.

Advantageously, a treatment method includes the step of administering antibodies to a patient with PIOL.

Advantageously, the antibody used in an embodiment is administered with a pharmaceutically acceptable carrier, suitable for the desired therapeutic effect.

In this regard, the antibody used in an embodiment may be used in combination with one or several other(s) antibodies, for example monoclonal(s), directed against one or more other(s) antigen(s) expressed on the lymphoid cells, such as, in a non exhaustive way, CD1, CD2, CD3, CD4, CD8, CD11, CD16, CD18, CD19, CD21, CD22, CD23, CD25, CD26, CD29, CD30, CD31, CD40, CD43, CD44, CD45, CD49, CD50, CD52, CD53, CD54, CD55, CD58, CD59, CD69, CD70, CD71, CD80, CD81, CD82, CD86, CD95, CD103, CD118, CD119, CD120, CD132, CD210, CD217 antigens.

In an embodiment, an antibody may be used in combination with cells that express Fc R such as NK cells, NKT cells (natural killer T), T lymphocytes, macrophages, monocytes or dendritic cells, that is to say, in combination with a cell therapy (Peller S, Kaufman S Blood 1991, 78:1569 ([8], which is incorporated by reference); Kimby E et al. 1989 Leukemia 3(7) :501-504 ([9], which is incorporated by reference); Soorskaar D et al. 1988 Int Arch Allery Appl Immunol 87(2), 159-164 ([10], which is incorporated by reference); Ziegler H W et al. Int J Cancer 1981 27(3), 321-327 ([11], which is incorporated by reference); Chaperot L et al. 2000 Leukemia 14 (9): 1667-1677 ([12], which is incorporated by reference); Vuillier F, Dighiero G 2003 Bull Cancer. 90(8-9):744-50([13], which is incorporated by reference)).

In addition, the antibody may preferably be administered to patients at a dose of less than approximately 375 mg/m², approximately 187.5 mg/m², approximately 75 mg/m², of approximately 37.5 mg/m², approximately 15 mg/m², approximately 7.5 mg/m² or less than approximately 3.75 mg/m² or approximately 1 mg/m² or approximately 0.5 mg/m². The dose administered may be approximately between 187.5 mg/m² and 75 mg/m², or approximately between 75 mg/m² and 37.5 mg/m², or approximately between 75 mg/m² and 15 mg/m², or approximately between 75 mg/m² and 7.5 mg/m² or approximately between 75 mg/m² and 3.75 mg/m². The administered dose may be approximately between 3.75 mg/m² and 0.5 mg/m², these doses are approximately between 2 mg/m² and 1 mg/m². These doses are administered intravenously.

When the antibody is administered intravitreally, the amount of antibody administered to the patient can be approximately between 0.001 μg and 1000 μg antibodies, or approximately between 0.001 μg and 100 μg, or approximately between 10 μg and 100 μg, approximately between 0.01 μg and 10 μg antibodies, or approximately between 1 μg and 10 μg, or approximately between 0.01 μg and 1 μg, or approximately between 0.01 μg and 0.1 μg or approximately between 0.02 μg and 0.08 μg.

The administration of the antibody may be carried out in the eye affected by the disease.

The amount of antibody administered to the patient can be approximately between 0.001 μg and 1000 μg of antibodies, or approximately between 0.001 μg and 100 μg of antibodies, or approximately between 10 μg and 100 μg of antibodies, for example approximately between 0.01 μg and 10 μg of antibody, or approximately between 1 μg and 10 μg, or approximately between 0.01 μg and 1 μg, or approximately between 0.01 μg and 0.1 μg or approximately between 0.02 μg and 0.08 μg of antibody per eye.

A patient may be administered approximately between 0.1 μg and 1000 μg of antibody/mL of vitreous humor, or approximately between 0.1 μg and 100 μg of antibody/mL of vitreous humor, or approximately between 10 μg and 100 μg of antibody/mL of vitreous humor, for example approximately between 1 μg and 100 μg of antibody/mL of vitreous humor, or approximately between 100 micrograms and 1000 micrograms/mL of vitreous humor, or approximately between 1 μg and 100 μg/mL of vitreous humor, or approximately between 1 μg and 10 μg or approximately between 2 μg and 8 μg of antibody/mL of vitreous humor.

The administration may be either through a single dose or a repeated dose injection. Alternatively, each administration may be sufficiently spaced from the previous administration so that each administration is considered as a single dose administration. For example, each administration may be at least at approximately a week interval, or at least at approximately 2 weeks, or at least at approximately 3 weeks, or at least at approximately 4 weeks, or at least at approximately 6 weeks, or at least at approximately 10 weeks, or at least at approximately 6 months.

The administration of the antibody may be achieved intravitreally with a quantity of approximately 0.02 μg of antibodies per eye or approximately 0.002 mg of antibody/ml of vitreous humor. Each administration is spaced by minimum of approximately a week, for example, approximately 1 week, approximately 2 weeks, approximately 3 weeks, approximately 4 weeks or approximately 5 weeks.

The mode of administration of the antibody used in an embodiment may be intraocular, for example, by injection into the vitreous body, by peri- and intra-ocular injection, by subconjunctival injection, by peri-or latero-bulbar injection, by retrobulbar injection, by instillation of eye drops, particularly intravitreal instillation, by iontophoresis, by intrascleral implantation, by application of an eye ointment, by retinal intravenous injection or by application of ultrasounds.

The antibody may be administered as a drug for treating lymphomas, in accordance with the standard rules which are well known.

The antibodies used in an embodiment have the feature and advantage of being cytotoxic.

As such, they have the ability to activate, in vivo and in vitro, by their Fc region the Fc RIIIA receptor. This is of considerable interest because this receptor is expressed at the surface of cells called “effector cells”: the binding of the Fc region of the antibody to its receptor carried by the effector cell causes the activation of Fc RIIIA and the destruction of the target cells. The effector cells are for example NK (Natural Killer) cells, macrophages, neutrophils, CD8 lymphocytes, T lymphocytes, NKT cells, eosinophils, basophils or the mast cells.

An embodiment is the use of an antibody for the manufacture of a drug for the treatment of primary intraocular lymphoma.

Moreover, an embodiment is a method for preparing a drug for the treatment of primary intraocular lymphoma, consisting in mixing the monoclonal antibody with a pharmaceutically acceptable support, then in obtaining the drug.

An embodiment is a method of therapeutic treatment of primary intraocular lymphoma including the administration, to a patient presenting the need, of an antibody or a drug such as described above.

Other advantages may appear to one upon reading the following examples, illustrated by the accompanying figures, given for illustrative purposes.

### BRIEF DESCRIPTION OF THE DRAWINGS

FIG. 1 is the schematic representation of the CKHu vector used for the chimerization of the light chain kappa of EMAB603 antibodies according to an embodiment.

FIG. 2 is the schematic representation of the G1Hu vector used for the chimerization of the heavy chain of EMAB603 antibodies according to an embodiment.

FIG. 3 is the schematic representation of the expression vector of the heavy and light chains used for the production of the EMAB603 antibody according to an embodiment.

FIG. 4 illustrates the absolute number of the tumor ocular cells (×10-⁶) in PIOL murine models after injection of PBS 1×, the LFB_R603 antibody (20 μg) or the Rituximab antibody (20 μg) in the eye that developed the tumor according to an embodiment. The results are from a pool of two experiments.

FIG. 5 illustrates the absolute number of the ocular tumor cells (×10-⁶) in PIOL murine models after injection of buffer LFB_R603 antibody or the LFB R603 antibody at doses of 0.02 μg and 0.2 μg in the eye that developed the tumor according to an embodiment. The results come from a pool of six experiments.

### DETAILED DESCRIPTION

### EXAMPLES

### Example 1

### Construction of Expression Vectors of the Anti-CD20 Chimeric Antibody EMAB603

A. Determination of the Sequence of the Variable Regions of the Murine CAT—13.6E12 Antibody

The total RNA of the murine CAT-13.6E12 hybridoma (provider: DSMZ, ref.ACC 474) producing an immunoglobulin of type IgG2a,_{K} has been isolated (kit RNAeasy, Qiagen ref. 74104). After reverse transcription, the variable fields of the light (VK) and heavy (VH) chains of the CAT-13.6E12 antibody have been amplified by the 5′RACE technique (Rapid Amplification of cDNA Ends) (kit GeneRacer, Invitrogen ref. L1500-01). The primers used for these two steps are the following:

1. reverse transcription primers
a. Kappa murine specific reverse primer
(SEQ ID NO: 9)
5′-ACT GCC ATC AAT CTT CCA CTT GAC-3′
b. G2a murine specific reverse primer
(SEQ ID NO: 10)
5′-CTG AGG GTG TAG AGG TCA GAC TG-3′
2. 5′RACE PCR primers
a. Kappa murine specific reverse primer
(SEQ ID NO : 11)
5′-TTGTTCAAGAAGCACACGACTGAGCAC-3′
b. G2a murine specific reverse primer
(SEQ ID NO: 12)
5′-GAGTTCCAGGTCAAGGTCACTGGCTCAG-3′

The resulting VH and VK PCR products were cloned in the pCR4Blunt-TOPO vector (Zero blunt TOPO PCR cloning kit, Invitrogen, ref.K2875-20) then sequenced. The nucleotide sequence of the region V_{K} of the murine antibody CAT-13.6E12 is indicated under sequence SEQ ID NO: 1, except for the last nucleotide which is replaced with A (AAA instead of AAC). The V_{K} gene belongs to the V_{K}4 family (Kabat et al., “Sequences of Proteins of Immunological Interest”, NIH Publication, 91-3242 (1991) [19], which is incorporated by reference). The nucleotide sequence of the region VH of CAT-13.6E12 is sequence SEQ ID NO: 2. The VH gene belongs to the VH1 family ([19]).

B. Construction of Expression Vectors of Heavy Chains and Light Chains of the Chimeric Antibodies EMAB603

1. Kappa Light Chain Vector of the Antibody EMAB603

The VK sequence cloned in the sequencing vector pCR4Blunt-TOPO was amplified using the following cloning primers

a) forward VK primer
(SEQ ID NO: 13)
5′-CTCAGTACTAGTGCCGCCACCATGGATTTTCAAGTGCAGATTTTCA
G-3′

The underlined sequence corresponds to the site of restriction Spe I, the sequence in bold corresponds to a Kozak consensus sequence, the ATG initiator is in italic.

b) reverse VK primer: (SEQ ID NO: 14)

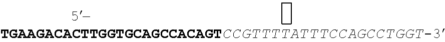

This primer achieves the junction of V_{K} murine sequences (in italic) and the human constant region (C_{K}) (in bold). The underlined sequence corresponds to the restriction site Dra III.

This primer achieves the junction of V_{K} murine sequences (in italic) and human constant region (C_{K}) (in bold). The underlined sequence corresponds to the restriction site Dra III.

The resulting product of V_{K} PCR contains the sequence coding the peptide, natural signal of the murine antibody CAT-13.6E12, with the mutation AAC→AAA (nucleotide in a box in the sequence of the reverse primer SEQ ID NO: 14) which corresponds to the mutation N106K with respect to the natural sequence V_{K} of CAT-13.6E12.

The sequence of the light chain of the chimeric antibody EMAB603 coded by this vector is presented in SEQ ID NO: 5 for the nucleotide sequence and corresponds to the deduced peptide sequence SEQ ID NO:7.

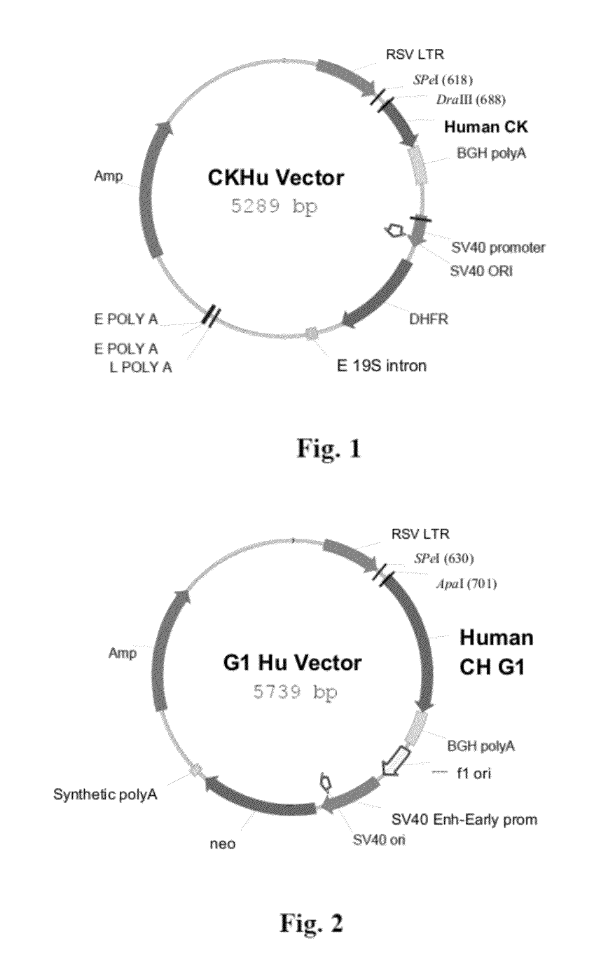

This V_{K} PCR has was then cloned between the sites Spe I and Dra III of the light chain chimerization vector (FIG. 1) which corresponds to the sequence SEQ ID NO: 1, in 5′ of the human constant region C_{K} the nucleic sequence of which is the sequence SEQ ID BO: 4. The human sequence V_{K} of this chimerization vector was modified beforehand by silent mutagenesis in order to create a restriction site Dra III in order to make it possible to clone V_{K} murine sequences. This chimerization vector contains an RSV promoter and a sequence of polyadenylation bGH (bovine Growth Hormone) as well as the dhf selection gene (dihydrofolate reductase).

2. Heavy Chain Vector

A similar measure has been applied for the chimerization of the heavy chain of the EMAB603 antibody.

The cloned VH sequence in the vector pCR4Blunt-TOPO was first amplified using the following cloning primers:

a) forward VH primer
(SEQ ID NO: 15)
5′-CTCAGTACTAGTGCCGCCACCATGGGATTCAGGATCTTTCTC-3′

The underlined sequence corresponds to the restriction site Spe I, the sequence in bold corresponds to a consensus Kozak sequence, the ATG initiator is in italic.

b) reverse VH primer
(SEQ ID NO: 16)
5′-GACCGATGGGCCCTTGGTGGAGGCTGAGGACGGTGACTGAGGTTCC-
3′

This primer achieves the junction of VH murine sequences (in italic) and constant GI human region (in bold). The underlined sequence corresponds to the restriction site Apa I.

The amplified VH fragment contains the sequence encoding the natural peptide signal of the murine CAT-13.6E12 antibody. This VH PCR was then cloned between the sites Spe I and Apa I of the heavy chain chimerization vector (FIG. 2) which corresponds to the sequence SEQ ID NO: 2, in 5′ of the human constant region I whereof the nucleic sequence is the sequence SEQ ID NO: 3. This chimerization vector contains an RSV promoter and a sequence of polyadenylation bGH (bovine Growth Hormone) as well as the neo selection gene.

The sequence of the heavy chain of the chimeric antibody EMAB603 coded by this vector is presented in SEQ ID NO: 6 for the nucleotide sequence and in sequence SEQ ID NO: 8 for the deduced peptide sequence.

3. Final Expression Vectors

Expression Vector of the Antibody EMAB603

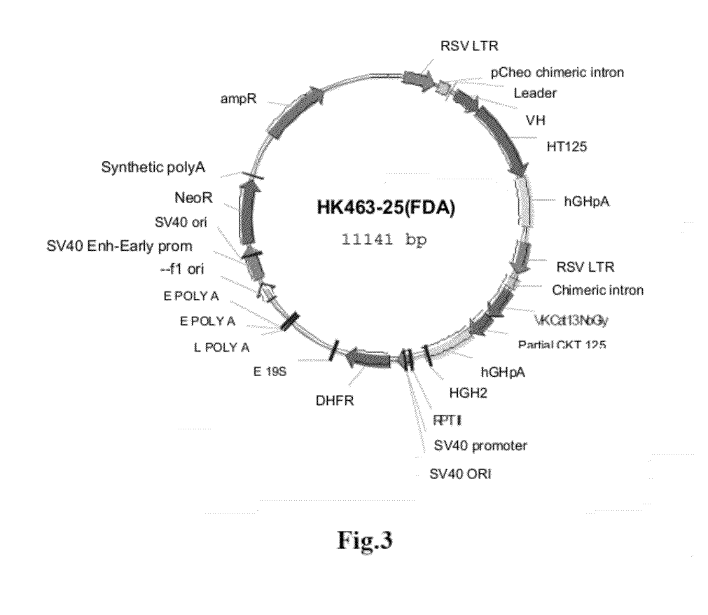

A unique expression vector containing two units of transcription of heavy chain and light chain of the anti-CD20 antibody EMAB-603 were constructed from the two chimerization vectors of the light chain and the chain. This expression vector HK463-25 (FDA) presents two selection genes, neo (neo-phosphotranspherase II) and dhfr (dihydrofolate reductase) as well as two units of transcription heavy chain and light chain under the control of an RSV promoter (FIG. 3).

### Example 2

### Generation of Cell Line Derivatives of the YB2/0 Line Producer of the Anti-CD20 Chimeric Antibody EMAB603

The rat line YB2/0 (ATCC #CRL-1662) has been cultivated in an EMS medium (Invitrogen, ref.041-95181M) containing 5% fetal calf serum (JRH Biosciences, ref.12107). For the transfection, 5 million cells have been electroporated (electroporator Biorad, model 1652077) in Optimix medium (Equibio, ref.EKITE 1) with 25 μg of light chain vector, pRSV-HL-EMAB-603 for the expression of the antibody EMAB603. The applied electroporation conditions were of 230 volts and 960 microfarads for a cuvette of 0.5 ml. Each electroporation cuvette was then distributed on 5 plates P96 with a density of 5000 cells/well. The placing in selective RPMI medium (Invitrogen, ref 21875-034) containing 5% dialysis serum (Invitrogen, ref. 1063-017), 500 μg/ml of G418 (Invitrogen, ref. 10131-027) and 25 nM of methotrexate (Sigma, ref.M8407) was achieved 3 days after the transfection.

The supernatants of the resistant transfection wells have been screened for the presence of chimeric immunoglobulin (Ig) by ELISA test specific to human Ig sequences.

The 10 transfectants generating the most antibodies have been amplified on P24 plates and their supernatant retested by ELISA in order to assess their productivity and to select the 3 best producers for the cloning by limiting dilution (40 cells/plate).

After the cloning, clone R603 was selected for generating the chimeric antibody EMAB603 and progressively adapted to the production medium CD Hybridoma (Invitrogen, ref. 11279-023). The production of chimeric antibodies EMAB603 has been achieved by the expansion of the line adapted in the CD Hybridoma medium, obtained by dilution at 3×10⁻⁵ cells/ml in bottles of 75 cm² and 175 cm² then by dilution at 4.5×10⁻⁵ cells/ml in roller-type bottles. After having reached the maximum volume, the line was pursued until the cell viability was only 20%. After production, the chimeric antibodies EMAB603 were purified by affinity chromatography on protein A (purity assessed by HPLC<95%) and monitored by poly-acrylamide gel electrophoresis.

### Example 3

### Study of the Effectiveness of LB_R603 and Rituximab Antibodies in a PIOL Murine Model Based on a First Experimental Protocol

Material and Methods

Cell Line

IIA1.6 cells are derived from the lymphomatous B murine A20-nJ line (Jones C. et al. “Different phenotypic variants of the mouse B cell tumor A20/2J are selected by antigen- and mitogen-triggered cytotoxicity of L3T4-positive, I-A-restricted T cell clones. J. Immunol. 1986;136-348-356, [21], which is incorporated by reference). The cells are cultivated in an RPMI medium (Roswell Park Memorial Institute medium, Glutamax; Invitrogen-Gibco, Cergy Pontoise, France), supplemented with 10% fetal calf serum (FCS; PAA laboratories, Colbe, Germany), penicillin 100 U:mL, and streptomycin 100 μg:mL (Eurobio, Les Ulis, France), 10 mM of sodium pyruvate (Invitrogen-Gibco), and 50 mM of mercaptoethanol (Invitrogen-Gibco), and are maintained at 37° C. with 5% of CO2 (Touitou et al. “Impaired Th1/Tc1 cytokine production of tumor-infiltrating lymphocytes in a model of primary intraocular B-cell lymphoma, investigative Ophtalmology & Visual Science, July 2007, vol. 48, no. 7, [22], which is incorporated by reference).

Transfection

IIA1.6 cells are transfected by nucleofection, by means of a plasmid of 3.5 kb pmax GFP (Amaxa Biosystems, Cologne, Germany), under the control of cytomegalovirus (CMV) promoter, and by means of a plasmid carrying the coding gene for the human CD20. When they are illuminated with an Argon laser at 488 nm, the GFP molecules (Green Fluorescent Protein) emit in the green wavelengths at 510 nm and make it possible to detect transfected cells in vivo. After transfection, the cells are cultivated in a medium of line containing 0.5 mg/ml of neomycin (G418). The clones expressing high ratios of GFP and human CD20 (hCD20) are obtained by limiting dilution and are named A20.IIA-GFP-hCD20.

Mice

Female mice BALB/c (H2^{d}) aged between 6 to 12 weeks are obtained by the Charles River laboratories (L'Arbresle, France). The mice are fed ad libitum with sterile food and filtered water, and held in cycles of 12 hours in black light. All mice are handled according to the European Union Guidelines and the “ARVO Statement for the Use of Animals in Ophtalmic and Vision research”.

Intravitreal Injections and Clinical Evaluations

The anesthesia is carried out by intraperitoneal injection of a combination of ketamine at 120 mg/kg (Imalgene 1000; Merial, Lyon, France) and xylazine at 6 mg/kg (Rompun 2%; Bayer, Leverkusen, Germany). The tumor cells (104 cells) are incubated in 2 μL 1× of PBS (pH 7.4), and are injected intravitreally through the pars plana using a dissecting microscope.

The injection is carried out in aseptic conditions in the right eye after dilation with tropicamide at 0.5% (Théa, Clermond-Ferrand, France), through a 32-gauge needle attached to a syringe (Hamilton; Hamilton Bonaduz, Switzerland). The test mice are injected intravitreally 2 μL PBS in the right eye. Rifamycine drops (Merck, Sharp & Dohme-Chribert, Clermont-Ferrand, France) are instilled after the injection.

An exam by slit lamp is carried out at regular intervals, including a bilateral exam of the bottom of the eye of each mouse. The clinical progression is graduated according to a clinical score of eye involvement

7 days after the intravitreal injection of cells A20.IIA-GFP-hCD20, the mice are divided into 3 groups: a group of 8 mice receive an injection of PBS 1× (2 μL) in the eye having received the tumor cells, 16 mice receive an injection of antibodies LFB_R603 (also called “EMAB603”) at 20 μg/2 μL in the eye having received the tumor cells, and 8 mice receive an injection of the Rituximab antibody at 20 μg/2 μL.

8 days after the injection of PBS, of antibody LFB_R603 or of the Rituximab antibody, the mice are euthanized by cervical dislocation.

Histology

After death, the eyes are collected, fixed in 4% paraformaldehyde containing 5% sucrose for 2 hours, and embedded in a resin for thin-section histology according to the instructions of the supplier (Historesin embedding kit, Leica Microsystems, Heidelberg, Germany). The serial sections (5 μm) are labeled with blue toluidine. The microscopic exam of the section of the eyes is thus carried out (Leitz microscope; Aristoplan, Rueil-Malmaison, France). The images are collected (DFC480 Leica, with a IM20 Image manager software; Leica Microsystems).

Immunocytochemistry

The enucleated eyes are fixed in a solution containing 4% paraformaldehyde and 5% sucrose for 2 hours, then immersed for one night in PBS containing 15% sucrose. The samples are embedded and frozen in an adapted component (Tissue-Tek; Sakura Finetek, Zoeterwoude, The Nederlands) and stored at −80° C. The frozen anteroposterior sections (10 μm of thickness) of the eyes at the optical nerve are cut with a cryostat (CM2050S; Leica, Wetzlar, Germany) and mounted in slides coated with gelatin for the immunocytochemistry analysis. For the immunolabeling, the tissue sections are incubated with a purified rat monoclonal antibody (mAb) directed against T cells (clone CD4 GK1.5, clone CD8 53-6-7; BD Biosciences, Le Pont-de-Claix, France), macrophages (clone F4/80) and polynuclear neutophiles clone 7/4; Serotec, Cergy Saint-Christophe, France). The visualisation is carried out with an Alexa594 anti-mouse conjugate antibody (Invitrogen-Gibco).

In certain experiments, the slides are incubated with the nuclear labeling agent propidium iodide (Invitrogen-Molecular Probes, Eugene, Oreg.). The experiments of negative test are achieved by incubation in tissue sections with a mAb isotype control. The sections are mounted in PBS containing 5% glycerol and observed by fluorescence microscope (FXA, Microphot; Nikon, Melville, N.Y.).

Confocal Microscopy and Image Analysis

Confocal microscopy is achieved on sections of frozen eyes with a laser scanning confocal microscope (LSM510; Carl Zeiss Meditec, GmbH, Oberkochen, Germany), equipped with an argon laser (488 nm) and a helium-neon laser (543 nm) The images are blended with an image-browser software (LSM; Carl Zeiss Meditec, GmbH) to produce a multicolor composite image.

Flow Cytometry

The eyes are dissected in RPMI medium, digested with 0.1 mg/mL of Dnase I (Roche, Meylan, France) and 1.67 units of Wünch/mL of purified enzymes (Liberase, Roche) at 37° C. for 20 minutes, filtered and rinsed in PBS with 2 mM of EDTA and 3% FCS (fetal calf serum).

The cells are pre-incubated with 2.4G2 mAb (10 μg/mL) to block the non specific link with the Fc receptors then 10⁵ cells per well are labeled with the following mAbs: anti-CD3 conjugated to the biotin (145-2C11; BD Biosciences), anti-CD4 conjugated to the phycoerythrin (GK1,5; BD Biosciences), anti-CD8 conjugated to fluorochrome Cy-Chrome) (53-6.7; BD Biosciences), anti-CD19 conjugated to phycoerythrin (6D5; e-Bioscience, San Diego, Calif.), anti-CD20 conjugated to the phycoerythrin (LFB_R603, LFB SA) or the mAb isotype control correspondents (BD Biosciences).

The flow cytometry analyses (FACSCalibur) are achieved with CellQuest and FACS Diva (BD Bioscience) software.

Results

Intraocular development of a lymphoma of cells B in the intravitreal injections of cells A20.IIA-GFP-hCD20

In order to generate an intraocular lymphoma model, normal immunocompetent mice BALB/c (H2^{d}) receive an intravitreal injection of the syngenic line of cell lymphoma B A20.IIA-GFP-hCD20, expressing the human CD20. These cells also expressing GFP, it is possible to discriminate B cells from lymphomas (CD19⁺GFP⁺), B cells from normal host B cells (CD19⁺GFP⁻).

Cells B A20.IIA-GFP-hCD20 are detected by flow cytometry in all eyes having been inoculated, by double detection of the GFP and CD19. The percentage of intraocular lymphomatous cells is correlated in the dose-response form to the number of cells initially injected in the right eyes at an initial dose of 10⁴ cells. The results are reproducible, with the development of intraocular lymphoma in all eyes having received the injection with A20.IIA-GFP-hCD20 cells.

The model thus very closely mimes the human PIOL

Effectiveness of the antibody LFB_R603 to treat PIOL in vivo

In the PIOL model obtained through injection of lymphomatous B cells expressing the human CD20, 8 mice receive an injection with PBS 1×, 16 mice are treated with the LFB_R603 antibody (20 μg/2 μL), and 8 mice are treated with the Rituximab antibody (20 μg/2 μL).

The absolute number of tumor cells is measured in the eyes, for each group tested in a pool of 2 independent experiments called LC_{—}08 and LC_{—}09, carried out according to the protocol detailed below, and compared by the statistical test of Mann-Whitney.

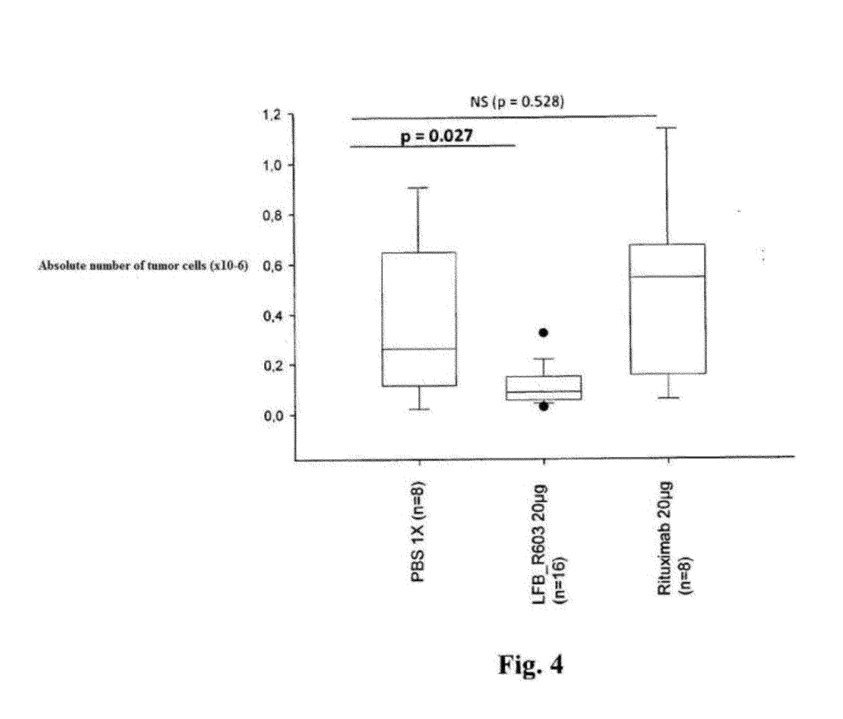

FIG. 4 represents the number of tumor cells amongst the total number of living cells after injection of PBS, or after treating by means of the LFB_R603 antibody or the Rituximab antibody.

The percentage of inhibition of tumor cell replication is given in table I.

| | PBS 1x | LFB_R603 | Rituximab |
|---|---|---|---|
| | (n = 8) | 20 μg (n = 16) | 20 μg (n = 8) |
| percentage of | — | 71.10% | NS |
| inhibition of tumor | | | |
| cell replication | | | |

Statistical test: Mann-Whitney

NS means: not significant

No inhibition is observed in mice having received PBS 1×.

No significant anti-tumor effect is observed in mice treated with the Rituximab antibody.

A significant anti-tumor effect is observed when mice are treated with LFB_R603 antibody.

### Example 4

### Study of the Effectiveness of LFB_R603 Antibodies and Rituximab in a PIOL Murine Model Based on a Second Experimental Protocol

Material and Methods

A PIOL method is obtained by the injection of lymphomatous B cells expressing the human CD20 in the eyes of mice in the detailed protocol of example 3.

In this second experimental protocol, 4 days after the intravitreal injection of cells A20.IIA-GFP-hCD20, the mice are divided into 3 groups: a group of 46 mice receive an injection with the antibody buffer LFB_R603, 16 mice are treated with the LFB_R603 antibody (0.02 μL/2 μL) and 32 mice are treated with the LFB_R603 antibody (0.2 μL/2 μL).

12 days after the injection of the LFB_R603 antibody buffer, of the LFB_R603 antibody at 0.02 μL/2 μL or of the LFB_R603 antibody at 0.2 μL/2 μL, the mice are euthanized by cervical dislocation.

Results

As detailed in example3, the mice develop an intraocular lymphoma of B cells, resulting from intravitreal injections of cells A20.IIA-GFP-hCD20 (thus expressing human CD20).

The absolute number of tumor cells is measured in the eyes having received the lymphoma cells, for each group tested in a pool of 6 independent experiments called LC_{—}12, LC_{—}14, LC_{—}15, LC_{—}16, LC_{—}19 and LC_{—}20, carried out according to the detailed protocol above, and compared with the statistical test of Mann-Whitney.

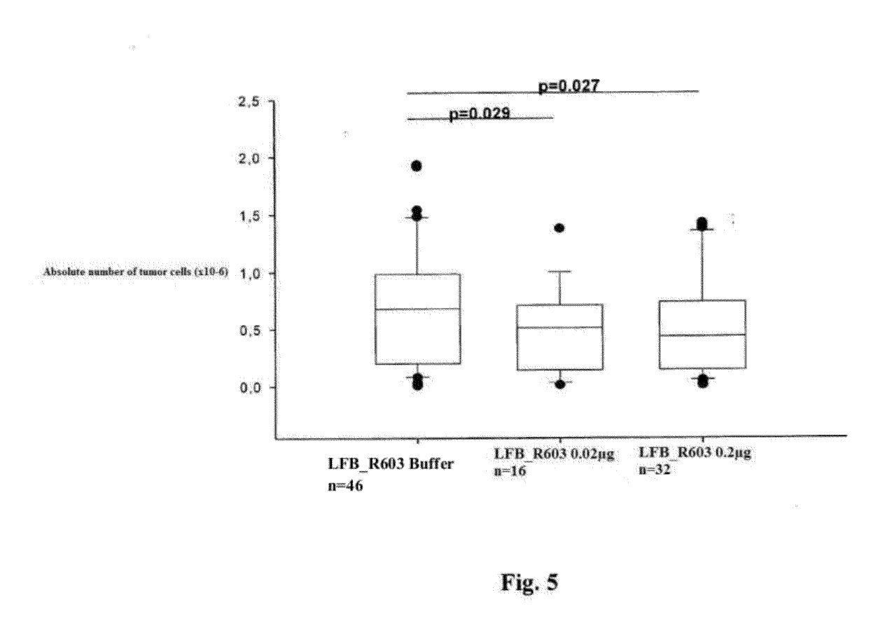

FIG. 5 represents the number of tumor cells amongst the total number of living cells after treatment by means of the antibody buffer LFB_R603, and the antibody LFB-R603 (0.02 μL/2 μL).

The percentage of inhibition of tumor cell replication is given in table II.

TABLE II

| | LFB_R603 | | |
|---|---|---|---|
| | Antibody buffer | LFB_R603 | LFB_R603 |
| | (n = 46) | 0.02 μg (n = 16) | 0.2 μg (n = 32) |
| Percentage of in- | — | 29.38% | 23.16 |
| hibition of tumor | | | |
| cell replication | | | |

Statistical test: Mann-Whitney

NS means: not significant

The collecting of these experiments shows that the treatment using the LFB_R603 antibody has a significant anti-tumor effect 12 days after the treatment even if injected at low concentration (e.g., 0.02 μg/2 μL and (0.2 μg/2 μL).

From the foregoing it will be appreciated that, although specific embodiments have been described herein for purposes of illustration, various modifications may be made without deviating from the spirit and scope of the disclosure. Furthermore, where an alternative is disclosed for a particular embodiment, this alternative may also apply to other embodiments even if not specifically stated.

### LIST OF REFERENCES

[1] Valentine et al. (1987); Proc Natl Acad Sci USA. 84 (22): 8085-9.

[2] Valentine et al. 1989 J. Biol. Chem. 264 (19): 11282-11287.

[3] Golay et al. (1985) J. Immunol.; 135(6):3795-801.

[4] Tedder et al. (1986) J. Immunol. 1986 August; 16(8):881-7.

[5] Teeling et al. (2004) Blood 104(6).-1793-800.

[6] Ohguro et al. (2008). Arch. Ophtalmol/Vol. 126 (No. 7); 1002-1003.

[7] Kitzamnn et al. (2007) Eye 21, 1524-1527.

[8] Peller S, Kaufman S Blood 1991, 78:1569.

[9] Kimby E et al. 1989 Leukemia 3 (7): 501-504.

[10] Soorskaar D et al. 1988 Int Arch Allery Appl Immunol 87(2); 159-164.

[11] Ziegler H W et al. 1981 Int J Cancer 27(3);321-327.

[12] Chaperot L et al. 2000 Leukemia 14 (9):1667-77.

[13] Vuillier F, Dighiero G 2003 Bull Cancer. 90 (8-9):744-50.

[14] The Journal of the American Medical Association, 199, 519 (1967)

[15] Science, 122, 501 (1952)

[16] (Virology, 8, 396 (1959) [16])

[17] Proc. Natl. Acad. Sci. USA, 53, 288 (1965)

[18] J. Experimental Medicine, 147, 923 (1978)

[19] Kabat et al., “Sequences of Proteins of Immunological Interest”, NIH Publication, 91-3242 (1991)

[20] SMET et al. Bull. Soc. Beige Ophtalml., 279, 91-95, 2001

[21] Jones C. et al. <<Different phenotypic variants of the mouse B cell tumor A20/2J are seletced by antigen- and mitogen-triggered cytotoxicity of L3T4-positive, I-A-restricted T cell clones. J. Immunol.

1986; 136-348-356

[22] Touitou et al. <<Impaired Th1/Tc1 cytokine production of tumor-infiltrating lymphocytes in a model of primary intraocular B-cell lymphoma, Investigative Ophtalmology & Visual Science, July 2007, vol. 48, no. 7

All of the above references are incorporated by reference.

1. A monoclonal antibody directed against the CD20 antigen, in which the variable region of each of the light chains is coded by murine nucleic acid sequence SEQ ID NO: 1, in which the variable region of each of the heavy chains is coded by murine nucleic acid sequence SEQ ID NO: 2, and in which the constant regions of the light chains and of the heavy chains originate from a non-murine species for treating primary intraocular lymphoma.

2. The antibody according to claim 1, in which the constant regions of each of the light chains and each of the heavy chains are constant human regions.

3. The antibody according to claim 1, in which the constant region of each of the heavy chains is coded by the human nucleic acid sequence SEQ ID NO: 3 and in which the constant region of each of the light chains is coded by the human nucleic acid sequence SEQ ID NO: 4.

4. The antibody according to claim 1, in which each of the light chains is coded by the murine-human chimeric nucleic acid sequence SEQ ID NO: 5, and in which each of the heavy chains is coded by the murine-human chimeric nucleic acid sequence SEQ ID NO:6.

5. The antibody according to claim 4, in which each of the light chains is composed of the amino acid sequence SEQ ID NO: 7 and in which each of the heavy chains is made up by SEQ ID NO: 8.

6. The antibody according to claim 1, produced in the rat hybridoma YB2/0 (cell YB2/3HL.P2.G11.16Ag.20, registered at the American Type Culture Collection under number ATCC CRL-1662.)

7. The antibody according to claim 5, produced by clone R603 registered under registration number CNCM I-3529 in the national collection of cultures of microorganisms (CNCM).

8. Use of an antibody according to claim 1, for the manufacture of a medicament intended for treating primary intraocular lymphoma.

9. A therapeutic method for treating primary intraocular lymphoma, comprising administrating to a patient in need thereof, said antibody or a medicament comprising said antibody, such as described in claim 1.
